(19) United States
(12) Patent Application Publication
(10) Pub. No.: US 2005/0064419 A1
(43) Pub. Date: Mar. 24, 2005

(54) Method for genotyping microsatellite dna markers by mass spectrometry

(76) Inventors: Abdelmajid Belouchi, Quebec (CA); Diane Saint-Louis, Quebec (CA); Bruno Paquin, Quebec (CA)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/493,401

(22) PCT Filed: Oct. 7, 2002
(86) PCT No.: PCT/IB02/04157

(30) Foreign Application Priority Data
Oct. 26, 2001 (US) 60355068

(51) Int. Cl. C12Q 1/68; C12P 19/34

(52) U.S. Cl. 435/6; 435/91.2

(57) ABSTRACT

The present invention relates to a method for enotyping microsatellite DNA markers. The described protocol is to convert a double-strand PCR fragment encoding the microsatellite of interest into a single-stranded DNA fragment of approximately 30-50 nucleotides. The produced fragment comprises the repeated region with a few flanking nucleotides and is suitable for analysis by mass spectrometry.

### FIELD OF THE INVENTION

The present invention relates to a method for genotyping microsatellite DNA markers. Specifically, the present invention provides a method for distinguishing allele content in repeated DNA by converting a double-stranded PCR fragment encoding the microsatellite to a single-stranded DNA containing the repeated region with few flanking bases. Although the resulting products can be analyzed by gel electrophoresis, the main advantage of the present invention is that the products are sufficiently small to be analyzed by mass spectrometry.

### BACKGROUND OF THE INVENTION

The analysis of variation among polymorphic DNA provides valuable tools for genetic studies in the development of genetic engineering, medicine, gene mapping and drugs. For example, variations in polymorphic DNA allow one to distinguish one individual of a population from another, or to assess the predisposition of an individual to a heritable disease or trait.

Two types of genetic markers widely used in genetic studies include microsatellites and single nucleotide polymorphisms (SNPs). Microsatellites are genomic regions that are distributed approximately every 30 kilobases throughout the genome and that contain a variable number of tandemly repeated sequences of mono, di-, tri-, tetra-, penta-, hexa-, hepta-, octa- or nona-nucleotides. SNPs are found approximately every kilobase in the genome.

SNPs and microsatellites differ in primary DNA structure, relative genome density and genetic information. For example, SNPs are more suitable for genotyping with a high-density of markers than microsatellites because of their distribution and the high specificity of the SNP flanking sequence. Yet, microsatellites are more informative than SNPs because microsatellites typically possess four to sixteen different alleles compared to two alleles for SNPs.

Presently, the most commonly used methods for genotyping microsatellite markers are gel-based PCR fragment analysis (reviewed in Shi et al., 1999). Methods based on differential hybridization, are limited by the sequence identity of the microsatellite markers (see Korkko et al. 1998). Oligonucleotide Ligation Assays (OLAs) have also been used to genotype mono- and di-nucleotide repeated microsatellite markers (Zirvi et al., 1999a, 1999b, U.S. Pat. No. 6,054,564, WO 98/03673, EP956359). Recently, a high-throughput, cost-effective OLA was designed and reported to be applicable to mono-, di-, tri-, tetra-, penta-, hexa-, hepta-, octa- and nona-nucleotide repeated microsatellites (U.S. Ser. No. 09/840,717).

Mass spectrometry (MS) is an emerging tool for genotyping and it is well established for the genotyping of SNPs (reviewed in Jackson et al. 2000, see also U.S. Pat. No. 6,197,498). It has been successfully used for genotyping microsatellites as well (e.g. Wada et al. 1999, Hahner et al. 2000, U.S. Pat. No. 5,869,242), simply by comparing the size of PCR-amplified fragments containing the microsatellite. However, there is a limitation in the use of MS for genotyping microsatellites because PCR fragments of 100 bp and higher give poor resolution. This is a serious limitation because it is not always possible to amplify a short fragment containing a microsatellite repeat due to the low sequence specificity of the microsatellite flanking regions. The present invention yields single-stranded DNA fragments containing the repeated region and a few nucleotides from the flanking sequences that approximate 30-50 bases, independent of the size of the initial PCR fragment. Therefore, the resulting products of the present invention are more prone to reliable mass spectrometry analysis than PCR fragments.

The citation of any reference herein should not be construed as an admission that such reference is available as “Prior Art” to the instant application.

### SUMMARY OF THE INVENTION

The present invention relates to a method for genotyping microsatellite DNA markers. The advantage of the present invention is to produce single-stranded DNA fragments whose lengths allow MS analysis with good resolution. MS has become a valuable tool for high-throughput SNP genotyping (reviewed in Jackson et al. 2000, U.S. Pat. No. 6,197,498) but its application to genotyping microsatellites have been hampered by the DNA fragment size limit inherent to the technology. Indeed, it is necessary to produce DNA fragments of 100 nucleotides or less in order to get a good resolution in MS analysis (Little et al. 1995, Wada et al. 1999). The recent developments in using MS to genotype microsatellites involved the characterisation of small PCR fragments encoding the microsatellites. The protocols for the purification of small PCR fragments involve the affinity purification of the PCR product and release of one strand (Ross and Belgrader 1997), the magnetic purification of the PCR fragment and analysis of both strands (Hahner et al. 2000) and the uses of nested PCR followed by thorough purification (Wada et al. 1999), the hammerhead-mediated cleavage of a transcribed microsatellite template (Krebs et al. 2001) among others (see also U.S. Pat. No. 5,869,242).

Although these techniques produce reliable results, there are inherent disadvantages associated with each of them. They either involve the use of two PCR reactions (Wada et al. 1999), biotinylated primers (Ross and Belgrader 1997), costly purification kits (Hahner et al. 2000), the analysis of double-stranded DNA fragments (Wada et al. 1999, Hahner et al. 2000) or the transcription of a microsatellite-containing PCR fragment with subsequent hammerhead-mediated cleavage (Krebs et al. 2001), which limits their application in multiplexing. In addition, for genome wide analysis, it may not be possible to amplify all the microsatellites in PCR fragments smaller than 100 base pairs because of the low specificity of the flanking sequences of microsatellite markers.

The present invention yields single-stranded DNA fragments that approximate 50 nucleotides, independent of the size of the initial PCR fragment, thus making them suitable for reliable MS analysis. Moreover, in the present invention regular oligonucleotide primers can be used, thus keeping the cost of oligonucleotide synthesis to its minimum.

The first step of the present invention consists of amplifying a genomic region comprising a microsatellite DNA marker, using a 2′-deoxynucleoside 5′-triphosphate (dNTP) mix in which the 2′-deoxythymidine 5′-triphosphate (dTTP) is replaced by 2′-deoxyuridine 5′-triphosphate (dUTP), a thermostable DNA polymerase with its buffer, the appropriate combination of oligonucleotide primers and genomic DNA as a template. The resulting PCR fragment does not contain thymidine nucleotides, except in the oligonucleotide primers, but comprises instead uridine nucleotides. The amplified fragment is then treated with uracyl-DNA-glycosylase (UDG), which removes uracyl bases in single- or double-stranded DNA thus creating abasic sites (Duncan 1981). The uracyl-free DNA is then treated with an agent that cleaves abasic sites, preferably AP-endonucleases (Grossman & Grafstrom 1982, Bailly & Verly 1989, Doetsch & Cunningham 1990), chemical agents such as piperidine (Stuart & Chambers 1987), or strong bases (Grossman & Grafstrom 1982), among others (see Doetsch & Cunningham 1990 and Steullet et al. 1999 for other examples). The end product is a single-stranded DNA fragment that contains the repeated region of the microsatellite and a few flanking nucleotides (up to the first thymidine in the original sequence) (FIG. 1).

The principle described in the embodiment above holds for a repeated DNA region that contains thymidine on one strand only, e.g. CA- or CAG-repeated DNA. However, one skilled in the art can apply the same principle using another target nucleotide for the generation of abasic sites. In an embodiment suitable for use in a repeated DNA region that contain guanosines on only one strand (CAG- or ATC-repeated DNA), one can specifically target guanosines by treating the DNA fragment with dimethyl sulfate (DMS), which modifies guanosines. The resulting DNA may be treated with piperidine, which simultaneously removes the modified guanosines and cuts the abasic sites (Maxam & Gilbert 1977). In an alternative embodiment, the initial PCR fragment is treated with hydrazine in the presence of salt (sodium chloride, NaCl). This modifies the cytosines, which can be removed by treatment with piperidine, resulting in the concomitant cleavage of the abasic site (Maxam & Gilbert 1977). This embodiment is suitable in cases where the repeated DNA contains cytosines on only one strand. Other alternatives using the same principle are also contemplated. It is not necessary to use dUTP in the initial PCR reaction if the UDG is not being used.

In one embodiment, the present invention provides a method for genotyping different alleles of a microsatellite DNA marker wherein the amplified DNA is internally labelled using an alpha-radio-labelled deoxynucleotide during the amplification reaction. The resulting fragments, after the whole protocol, are separated by gel electrophoresis and the sizes of the fragments reflect the genotype of the sample. In a preferred embodiment, the amplified DNA is not radio-labelled and the resulting fragments, after the whole protocol, are analyzed by mass spectrometry.

The present invention further provides a method for genotyping a pooled DNA sample comprising a mixture of different DNA samples of the same microsatellite marker. The detection of the allele content of the microsatellite DNA marker within the pooled sample is determined by gel electrophoresis or mass spectrometry. In both cases, the signal is directly proportional to the concentration of the corresponding allele within the pooled DNA sample.

The present invention is likely to improve the signal to noise ratio needed to achieve genotyping of pooled DNA samples, and the throughput in large-scale genotyping projects.

### DESCRIPTION OF THE FIGURES

FIG. 1 shows a protocol used to produce single stranded DNA fragments. Panel 1 shows the putative genomic target. In this example, one allele of a CA-repeated microsatellite marker, [CA]₁₁ in bold, is shown with few flanking sequences. The boxed sequence constitutes the single-stranded DNA fragment that is going to be produced using the protocol described in the present invention. Panel 2 shows the sequence of the PCR fragment when amplified with a dNTP mix in which the dTTP had been replaced by dUTP. Uridine nucleotides are marked by arrows. Panel 3 shows the DNA treated with UDG. As shown in this panel, UDG has removed the uracyl from each uridine nucleotides. Dashes represent abasic sites. As discussed in the text, if another nucleotide than uridine is targeted, then the PCR reaction does not need to be performed using a dNTP mix containing UTP and the treatment by UDG is replaced by a chemical treatment. Panel 4 illustrates the results of the piperidine treatment. This reaction produces several single-stranded DNA fragments of various sizes. The main fragment contains the repeated region of the microsatellite marker with a few nucleotides from the flanking sequences. As pointed out in the text, one can use other abasic site-specific cleaving agents, such as AP-endonucleases or other chemicals. Panel 5 shows the products in order of their sizes, the larger product being the single-stranded DNA fragment used to genotype the locus.

FIG. 2 shows Example 1, which is the genotyping of the D6S471 locus. The D6S471 locus is a CA-repeated microsatellite DNA marker. Four samples were tested, each with a different genotype: a 13-14 CA heterozygous, a 13 CA homozygous, a 14 CA homozygous and a 16 CA homozygous. The stutter bands, which are one and two dinucleotides shorter, are clearly visible under the main bands. Fragments smaller than 17 nucleotides are not shown. Size markers (in nucleotides) on the left have been deduced from an unrelated sequencing reaction. Note that this is an approximation since the distance of migration varies depending upon the base composition of the DNA fragments.

FIG. 3 shows Example 2, which is the genotyping of the D6S273 locus. The D6S273 locus is a CA-repeated microsatellite DNA marker. Four samples were tested, each with a different genotype: a 17 CA homozygous, a 19 CA homozygous, a 17-19 CA heterozygous and an 18-21 heterozygous. The stutter bands are also visible under the main bands. Fragments smaller than 17 nucleotides are not shown.

FIG. 4 shows Example 3, which is the genotyping of the D6S1014 locus. The D6S1014 locus is a CAG-repeated microsatellite DNA marker. Five samples were tested, each with a different genotype: a 9 CAG homozygous, a 9-10 CAG heterozygous, a 9-12 CAG heterozygous, an 10-11 CAG heterozygous and a 6-10 CAG heterozygous. The sporadicity of the 16-nucleotide fragment, marked by an asterisk, is due to a sequence variation within the 22-nucleotide fragment. Fragments smaller than 16 nucleotides are not shown.

FIG. 5 shows the genotyping of a pooled sample of CA repeats. Two genomic DNAs having different genotypes at the D6S471 locus (13 CA homozygous and 14 CA homozygous) were mixed in various proportions prior to PCR amplification. The PCR products were then treated according to the protocol of the present description. Fragments smaller than 10 nucleotides are not shown.

FIG. 6 shows genotyping of a pooled sample of CAG repeats. Two genomic DNAs having different genotypes at the D6S1014 locus (9-12 heterozygous and 10-11 heterozygous) were mixed in various proportions prior to PCR amplification. The PCR products were then treated according to the protocol of the present description. Fragments smaller than 16 nucleotides are not shown.

FIG. 7 shows the mass spectrometry of a trinucleotide repeat. The DNA sample used in this example harbours a [CAG]₁₀ genotype at the D6S1014 locus. The two peaks at lower mass are DNA fragments produced by the flanking sequence. The peak at 10065 represents the repeat-containing fragment. The calculated masses of the fragments are shown in the inbox.

### DEFINITIONS

Numerous terms and phrases used throughout the instant Specification and appended Claims are defined below:

As used herein, the phrase “Abasic sites” refers to sites along the DNA molecule that are deprived of bases. The backbone, 2′-deoxyribose linked via 5′-3′ phosphodiester bonds, remains intact at these sites but the bases have been removed. The DNA can therefore no longer form base pairs at abasic sites.

As used herein, the phrase “Allele” refers to, at a given locus, a particular form of a gene or genotype, specifying one of all the possible forms of the character encoded by this locus. A diploid genome contains two alleles at any given locus.

As used herein, the phrase “AP endonucleases” refers to enzymes that recognize abasic sites and cleave the phosphodiester bond at such sites.

As used herein, the phrase “2′-deoxynucleoside 5′-triphosphate” refers to the triphosphate form of a nucleotide, also referred to as dNTP. DNA nucleosides are usually guanosine, adenosine, cytosine or thymidine. In this description, it also includes uridine.

As used herein, the phrase “2′-deoxyuridine 5′-triphosphate” refers to a DNA nucleotide in which the base is uracyl. Uracyl is capable of pairing with adenine.

As used herein, the phrase “Genotype” refers to a set of alleles at a specified locus.

As used herein, “Internal labelling” refers to a form of labelling in which the labels are attached within the DNA molecule as opposed to either 5 one of its ends. In this description, the PCR fragment can be internally labelled by using one or more of the 5′-[α-²²P]dNTPs during the course of the PCR amplification reaction.

As used herein, “Locus” refers to a specified region of the genome.

As used herein, “Microsatellite” refers to a DNA of eukaryotic cells comprising highly repetitive DNA sequences flanked by sequences unique to that locus. In this description, microsatellite refers to mono-, di-, tri-, tetra-, penta-, hexa-, hepta-, octa- or nona-nucleotide repeated regions.

As used herein, “Nucleotide” refers to a unit of a DNA molecule, that is composed of a base, a 2′-deoxyribose and phosphate ester(s) attached at the 5′ carbon of the deoxyribose. For its incorporation in DNA, the nucleotide needs to possess three phosphate esters but it is converted into a monoester in the process.

As used herein, “Oligonucleotide” refers to a short single-stranded deoxyribonucleic acid molecule. In this description, oligonucleotides are used as primers for the amplification reactions.

As used herein, “PCR (polymerase chain reaction) amplification” refers to an enzymatic process resulting in the exponential amplification of a specific region of a DNA template. The process uses a thermostable DNA polymerase, capable of replicating a DNA template from a primer. In the presence of two primers, the region between them is amplified following this process.

As used herein, “Pooled DNA sample” refers to an equimolar set of PCR fragments amplified from different individuals. The genomic region amplified is the same for all the fragments included in the pooled DNA sample.

As used herein, “Uracyl DNA Glycosylase (UDG)” refers to an enzyme that recognizes and removes uracyl bases in single- or double-stranded DNA, generating abasic sites at the locations where uridine nucleotides have been incorporated.

### DETAILED DESCRIPTION OF THE INVENTION

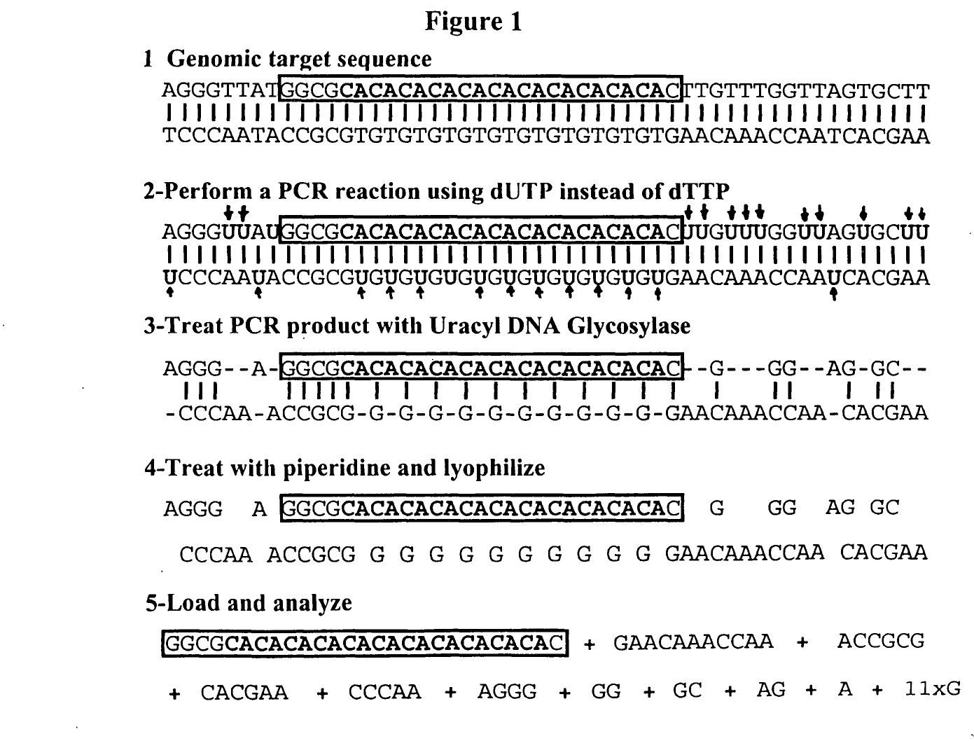

The present invention relates to a method for genotyping microsatellite DNA markers by mass spectrometry. MS has become a valuable tool for high-throughput SNP genotyping (reviewed in Jackson et al. 2000, U.S. Pat. No. 6,197,498) but its application to genotyping microsatellites have been hampered by the DNA size fragment limit inherent to the technology. It is preferable to produce DNA fragments of 100 nucleotides or less in order to obtain a good resolution in MS analysis (Little et al. 1995, Wada et al. 1999). The present invention is a protocol to produce single-stranded DNA fragments that include the repeated region of a microsatellite and approximately 30-50 nucleotides, independent of the size of the initial PCR fragments (FIG. 1). This protocol generates DNA fragments whose lengths allow MS analysis with good resolution in a cost-effective manner.

The present invention contains many advantages compared to existing protocols. The present invention does not require modified oligonucleotides, the template comprises a PCR fragment of any size encoding the microsatellite of interest, and it can be used to genotype pooled DNA samples. Also, the protocol in the present invention can be multiplexed as long as the size of the different microsatellite to genotype are of different sizes.

In one embodiment, the present invention comprises a genomic region containing the microsatellite of interest. The PCR reaction is performed using 2′-deoxyuridine 5′-triphosphate which replaces 2′-deoxythymidine 5′-triphosphate in the dNTP mix. The uridine nucleotide is incorporated as efficiently as the thymidine nucleotide during the amplification reaction at positions where a thymidine nucleotide would otherwise be incorporated (Slupphaug et al. 1993).

The uridine-containing DNA is then treated with Uracyl-DNA-Glycosylase. UDG removes uracyl bases in single- or double-stranded DNA, generating abasic sites at every position where a uridine nucleotide had been incorporated (Duncan 1981).

The DNA is then treated with a cleaving agent that is specific for abasic sites. This can be done both enzymatically and chemically. AP-endonucleases are enzymes that recognize and cut the DNA at abasic sites (Grossman & Grafstrom 1982, Bailly & Verly 1989, Doetsch & Cunningham 1990). Examples of AP-endonucleases include, but are not limited to the human AP-endonuclease (Fritz 2000), E. coli exonuclease III (Shida et al. 1996), endonuclease III (Bailly & Verly 1987) and endonuclease IV (Ramotar 1997). One skilled in the art can use any enzyme capable of recognizing and cutting abasic sites in DNA without altering the principle of the present invention. Some chemicals can also cleave the DNA at abasic sites, including but not limited to piperidine (Stuart & Chambers 1987), polyamines, intercalator amines, alkaline agents and other chemicals (Doetsch & Cunningham 1990, Steullet et al. 1999). One skilled in the art can use other chemicals that show the same properties without changing the principle of the present invention.

The protocol described above utilizing the dUTP can be applied to genotype microsatellites harbouring either A or T in the repeated sequences (e.g. CA-repeats). For microsatellites such as CA-repeats, the end-results of the protocol described in the present invention are single-stranded DNA fragments that approximate 30 to 50 nucleotides. These fragments include the repeated region plus some nucleotides from the flanking sequences. Since the DNA is cleaved at every position where a uridine nucleotide had been incorporated, one strand is completely degraded (the “TG” strand in this example) whereas the “CA” strand remains intact for the length of the repeat. The DNA is cut on both sides of the repeat on the “CA” strand at the sites where a uridine nucleotide had been incorporated (a thymidine nucleotide in the original DNA sequence).

The principle described above can be applied to genotype microsatellites harbouring either A or T in the repeated sequences (e.g. CA repeats) but not both (e.g. CAT repeats). Indeed, one skilled in the art can modify the protocol to suit other type of microsatellites by targeting different nucleotides, without changing the principle. This embodiment can for example analyze [ATC]-repeated microsatellites using the protocol described above by making the following adjustments. The locus is first amplified using regular protocols, without substituting dTTP by dUTP. The PCR fragment is then treated with either dimethyl sulfate (DMS), which modifies guanosines, or hydrazine in the presence of salt, which removes cytosines. The DMS- or hydrazine-treated DNA is then incubated in the presence of piperidine, which will remove the modified nucleosides and cut the abasic sites (Maxam & Gilbert 1977). In the two examples cited above, although the protocols are different than the one described in the present invention, the principle remains the same. It is obvious that one skilled in the art can envisage other alternatives using the same principle.

Unlike prior art protocols in which piperidine is used to partially modify the DNA (Maxam & Gilbert 1977, the DNA in the present invention is quantitatively modified, and the novel protocols disclosed herein produce short, single-stranded DNA fragments for the genotyping of microsatellite markers.

While U.S. Pat. No. 5,869,242, describes use of MS in the genotyping of microsatellites, the methods disclosed therein suffer from substantial limitations because the initial PCR fragment is analyzed as is, without pre-treating it to diminish its length. The use of dUTP in PCR and digestion of the PCR product by UDG is solely to decrease the molecular weight of the PCR fragment. Thus, only those initial fragments that are short enough in length can be meaningfully analyzed by MS. In contrast, the novel protocols disclosed herein yield a single-stranded DNA fragment of approximately 30-50 nucleotides that contains exclusively the repeated region with few flanking nucleotides. This short fragment can then be analyzed either by gel electrophoresis or MS. The present protocol provides the advantage of producing single-stranded DNA fragments that are in the high-resolution range of MS.

### EXAMPLES

The following non-limiting Examples serve to illustrate the genotyping of three loci; D6S273, D6S471 and D6S1014. D6S273 and D6S471 are dinucleotide [CA]-repeat microsatellite markers whereas D6S1014 is a trinucleotide [CAG]-repeat microsatellite marker. The use of polyacrylamide gel electrophoresis for size fragmentation of the products is a simple and temporary step for the improvement of the present technology. This entails the use of internally labelled PCR fragments for visualization of the end products upon exposure of the gel on an X-ray film. Ultimately, the products will be analyzed by MS and the PCR products will no longer need to be labelled.

In this description, we amplified the three loci, D6S273, D6S471 and D6S1014 from various individuals, using the oligonucleotides suggested by the NCBI human genome resources web site. The reaction conditions are those for regular PCR amplification (White 1993, U.S. Pat. No. 4,683,195) except that the 2′-deoxythymidine 5′-triphosphate in the dNTP mix has been removed and replaced by 2′-deoxyuridine 5′-triphosphate. Approximately 0.1 ul of [α-³²P)dCTP is added to the reaction mixture to internally label the amplified fragment.

The PCR fragment is then ethanol precipitated following regular protocols (Sambrook et al. 1989), and resuspended in 20 ul of distilled water. 10 ul of this solution (approximately 10-50 ng of a 100-200 bp fragment) is used for treatment with UDG. The UDG reaction is carried out in 50 ul with 5 units of UDG in its corresponding buffer as supplied by the manufacturer (New England Biolabs), for 30 minutes at 37° C. The DNA is then treated with piperidine by adding 40 ul of water and 10 ul of 10M piperidine (1M final concentration of piperidine, Sigma) and incubated for 30 minutes at 90° C. The DNA is then dried under vacuum. The dried pellet is resuspended in 100 ul of distilled water and dried again under vacuum. In order to completely remove the piperidine, the pellet is once again resuspended (in 20 ul) and dried. The samples are ready for analysis and resuspended in 1× loading buffer (95% formamide, 5 mM EDTA) and loaded on a 15% denaturing polyacrylamide gel for electrophoresis. As stated earlier, the objective of the present invention is to produce single-stranded DNA fragments that can be analyzed by MS. To this end, the PCR reaction does not need to be performed in the presence of radio-labelled nucleotides. However, a final, cleaning step is necessary for removing the salts before loading them on a mass spectrometer.

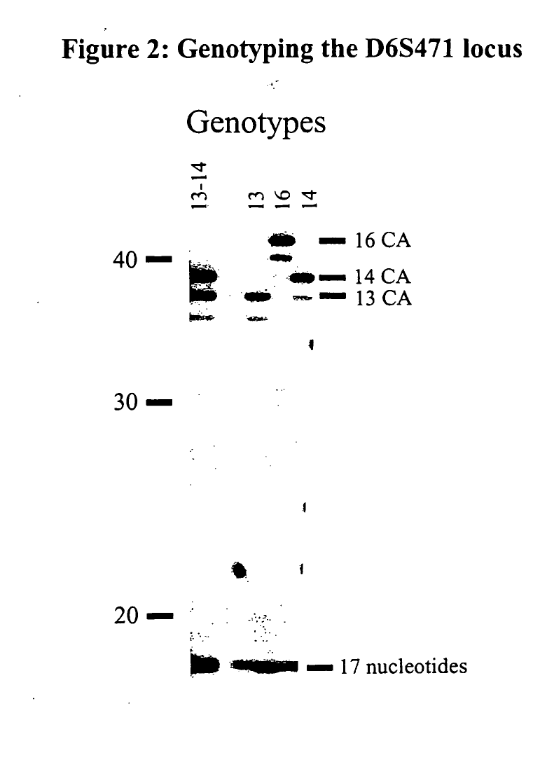

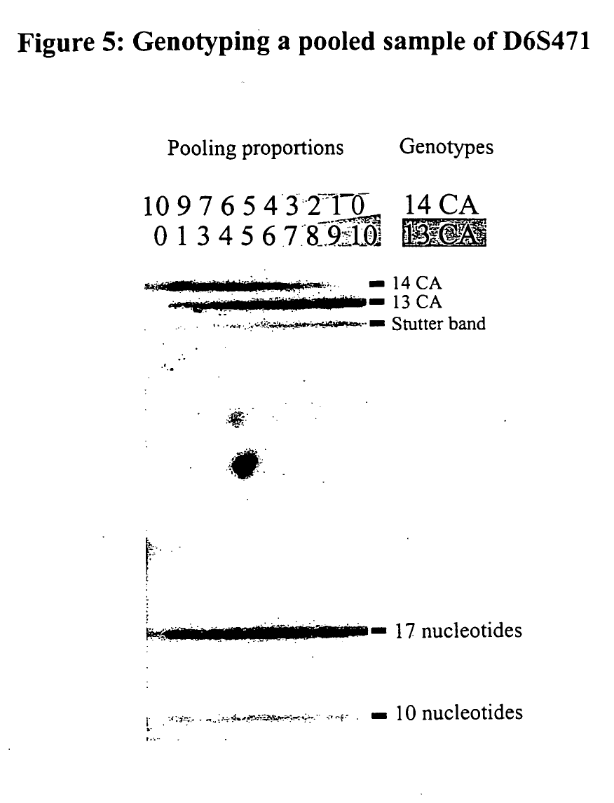

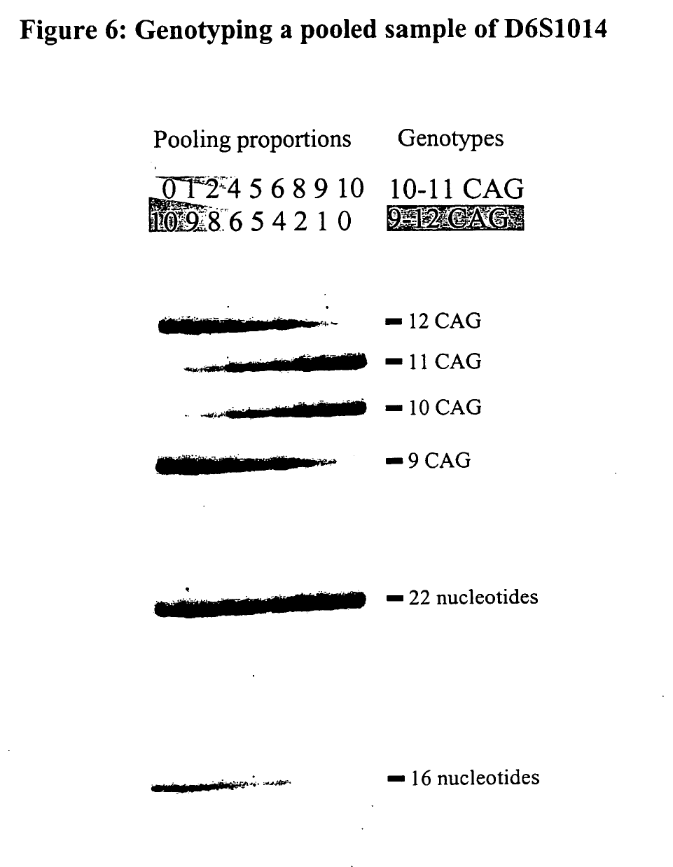

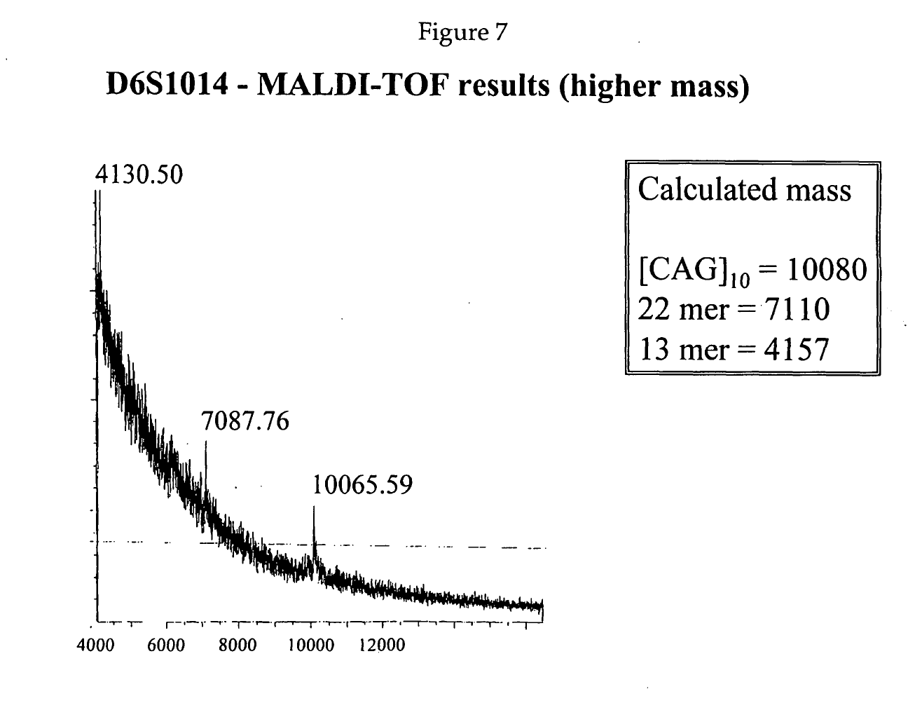

FIGS. 2-4 illustrate the results with three different microsatellite markers. Two examples are also performed on pooled DNA samples to demonstrate the feasibility of pooling using the present invention. The results of the pooling experiments are shown in FIGS. 5 and 6. FIG. 7 shows the analysis of a sample by MS.

### Example 1

### Genotyping the D6S471 Locus

First, the D6S471 locus was amplified and genotyped using the protocol described above. D6S471 is a CA-repeated microsatellite marker. There are four alleles in the population at this locus, [CA]₁₃, [CA]₁₄, [CA]₁₆ and [CA]₁₇. The PCR reaction yielded products between 107 and 116 bp depending upon the genotype of the sample. After treatments with UDG and piperidine, single-stranded DNA fragments of 37 to 45 bases are produced, comprising the [CA]-repeated region plus 11 nucleotides from the flanking sequences. Four individuals were genotyped at this locus, a 13 CA homozygous, a 14 CA homozygous, a 16 CA homozygous and a 13-14 CA heterozygous. These genotypes produce fragments of 37, 39, 43 and 37-39 bases, respectively, upon treatments with UDG and piperidine. As seen in FIG. 2, these fragments are indeed produced along with smaller fragments, 17 bases and lower, coming from the flanking sequences. The size markers on the left are approximate since the distance of migration may vary depending on the sequence of the fragments. The stutter bands appearing under the main bands are most likely the results of amplification artefacts and are the size on one or two dinucleotides shorter. It is well known that dinucleotide repeats are prone to amplification errors that can significantly compromise reliable allele designation (Schlotterer & Tautz 1992, Hauge & Litt 1993, Sprecher et al. 1996).

### Example 2

### Genotyping the D6S273 Locus

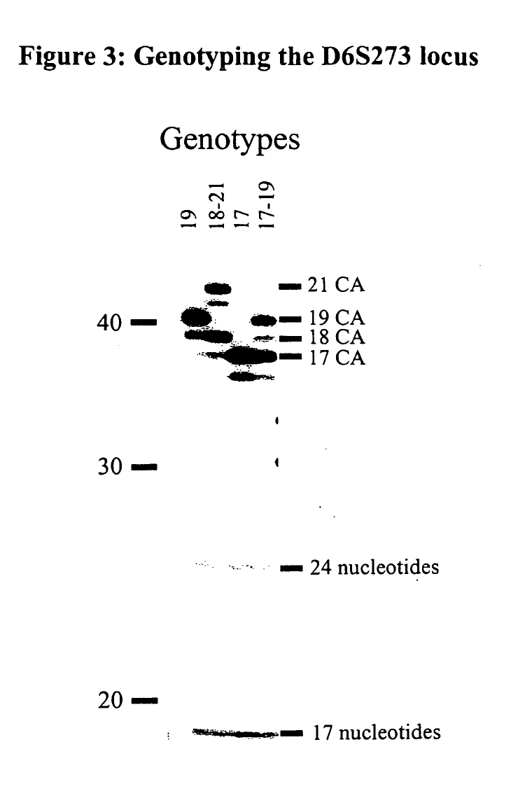

Second, the D6S273 locus was amplified and genotyped using the protocol described above. D6S273 is a CA-repeated microsatellite marker. There are 8 alleles in the population at this locus, [CA]₁₁ and [CA]₁₅ to [CA]₂₁. The PCR reaction yielded products between 120 and 140 bp depending upon the genotype of the sample. After treatments with UDG and piperidine, single-stranded fragments of 27 to 47 bases are produced, comprising the [CA]-repeated region plus 5 nucleotides from the flanking sequences. Four individuals were genotyped at this locus, a 17 CA homozygous, a 19 CA homozygous, a 17-19 CA heterozygous and an 18-21 CA heterozygous. These genotypes produce fragments of 39, 43, 39-43 and 41-47 bases, respectively, upon treatment with UDG and piperidine. As seen in FIG. 3, these fragments are indeed produced along with smaller fragments, 24 bases and lower, coming from the flanking sequences. The 24-base fragment comprises the reverse primer used in the PCR reaction elongated by three nucleotides.

### Example 3

### Genotyping the D6S1014 Locus

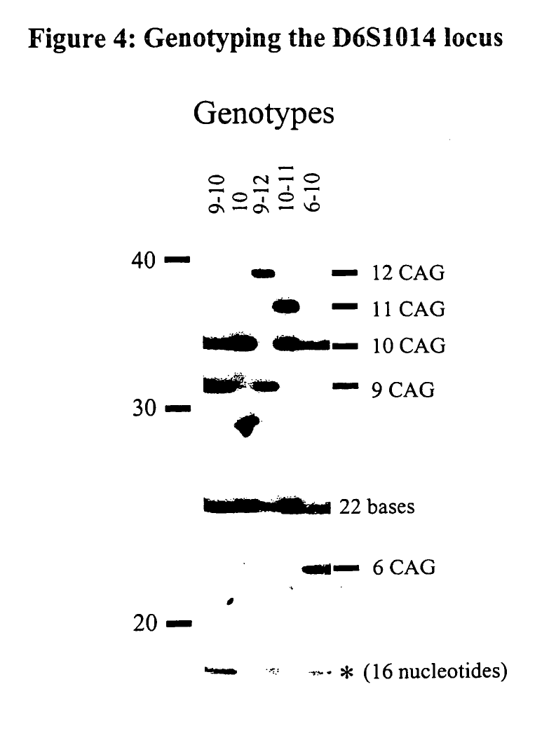

Third, the D6S1014 locus was amplified and genotyped using the protocol described above. D6S1014 is a CAG-repeated microsatellite marker. There are 6 alleles in the population at this locus, [CAG]₆ and [CAG]9 to [CAG]₁₃. The PCR reaction yielded products between 124 and 145 bp depending upon the genotype of the sample. After treatments with UDG and piperidine, single-stranded fragments of 20 to 41 bases are produced, comprising the [CAG]-repeated region plus 2 nucleotides from the flanking sequences. Five individuals were genotyped at this locus, a 10 CAG homozygous, a 9-10 CAG heterozygous, a 9-12 CAG heterozygous, a 10-11 CAG heterozygous and a 6-10 CAG heterozygous. These genotypes produce fragments of 32, 29-32, 29-38, 32-35 and 20-32 bases, respectively, upon treatment with UDG and piperidine. As seen in FIG. 4, these fragments are produced along with smaller fragments, 22 bases and lower, coming from the flanking sequences. The fragment of 22 bases that is produced from the flanking sequence does not represent a fragment specifying an allele. The intensity of the 22-nucleotide fragment varies along with the appearance of the 16-nucleotide fragment (marked with an asterisk). This is due to a nucleotide variation in the 22-nucleotide fragment that changes a C to a T, which under the conditions of the present protocol, yields two fragments of 5 and 16 nucleotides.

Pooling Experiments:

The pooling of DNA samples increases the throughput of the genotyping processes. However, it is preferable if the technology used is sensible enough to give an accurate ratio of the different alleles within the pooled sample. MS is capable of accurately calculating these ratios and therefore, the present invention can being used to genotype pooled DNA samples. By way of examples, two different pooled samples were tested.

### Example 4

### Pooling CA-Repeats

First, genomic DNA having homozygous genotypes at the CA-dinucleotide repeat D6S471 locus, a 13 CA and a 14 CA, were mixed in different proportions and submitted to PCR amplification and treated as described above. The results show that the ratios of the two alleles, as judged by the intensity of the signals, change according to the proportions of the DNA templates within the pool (FIG. 4).

### Example 5

### Pooling CAG-Repeats

Secondly, genomic DNA with different genotypes at the D6S1014 locus were mixed in different proportions and used as templates for PCR reactions and treatment with UDG-piperidine as described above. The genomic DNAs used in this pooling experiment had the 10-11 CAG heterozygous and the 9-12 CAG heterozygous genotypes. As with the previous experiment, the results show that the ratios of the alleles, as judged by the intensity of the signals, change according to the proportions of the DNA templates within the pool (FIG. 5).

### Example 6

### Mass Spectrometry

One of the samples was tested on a mass spectrometer. The PCR fragment was treated as above and the final products were cleaned using an ion exchange resin (Spectroclean from Sequenom). 10 nl were spotted on a Spectrochip (Sequenom) and the sample was analyzed on a linear Biflex III (Bruker Daltonics) on negative ion mode. The diagnostic peak could be observed at the expected mass. The peaks at lower masses are expected from the flanking sequences and appear at the expected masses.

It is understood that various other embodiments and modifications in the practice of the invention will be apparent to, and can be readily made by, those skilled in the art without departing from the scope of the invention described above. Accordingly, it is not intended that the scope of the claims appended hereto be limited to the exact description set forth above, but rather that the claims be construed as encompassing all of the features of patentable novelty which reside in the present invention, including all the features and embodiments which would be treated as equivalents thereof by those skilled in the art to which the invention pertains.

Various publications are cited herein, the disclosures of which are incorporated by reference in their entireties.

### REFERENCES

Bailly, V. & Verly, W. G. (1987) Biochem. J. 242: 565-572.
Bailly, V. & Verly, W. G. (1989) Nucleic Acids Res. 17: 3617-3618.
Doetsch, P. W. & Cunningham, R. P. (1990) Mutat. Res. 236: 173-201.
Duncan, B. K. (1981) in The Enzymes (Boyer, P. D., ed.) Academic Press, New-York, pp. 565-586.
Fritz, G. (2000) Int. J. Biochem. Cell Biol. 32: 925-929.
Grossman, L. & Grafstrom, R. (1982) Biochimie 64: 577-580.
Hahner, S., Schneider, A., Ingendoh, A. & Mosner, J. (2000) Nucleic Acids Res. 28: e82.
Hauge, X. Y. & Litt, M. (1993) Hum. Mol. Genet. 2: 411-415.
Jackson, P. E., Scholl, P. F. & Groopman, J. D. (2000) Mol. Med. Today, 6: 271-276.
Korkko, J., Annunen, S., Pihlajamaa, T., Prockop, D. J. & Ala-Korkko, L. (1998) Proc. Natl. Acad. Sci. USA, 95: 1681-1685.
Krebs, S, Seichter D & Forster M. (2001) Nat. Biotecnol. 19: 877-80.
   Little, D. P., Thannhauser, T. W. & McLafferty, F. W. (1995) Proc. Natl. Acad. Sci. USA 92: 2318-2322.
Maxam, A. M. & Gilbert, W. (1977) Proc. Natl. Acad. Sci. USA 74: 560-564.
Ramotar, D. (1997) Biochem. Cell Biol. 75: 327-336.
Ross, P. L. & Belgrader, P. (1997) Anal. Chem. 69: 3966-3972.
Sambrook, J., Fritsch, E. F. & Maniatis, T. (1989) Molecular cloning. Alaboratory manual, 2^{nd} ed. Cold Spring Harbor Laboratory press, Cold Spring harbor, N.Y.
Schlotterer, C. & Tautz, D. (1992) Nucleic Acids Res. 20: 211-215.
Shi, M. A., Bleavins, M. R. & de la Iglesia, F. A. (1999) Mol. Diagn. 4: 343-351.
Shida, T., Mitsuhiro, N. & Sekiguchi, J. (1996) Nucleic Acids Res. 24: 4572-4576.
Slupphaug G., Alseth I., Eftedal, I., Volden, G. & Krokan H. E. (1993) Anal. Biochem. 211: 164-169.
Sprecher, C. J., Puers, C., Lins, A. M. & Schumm, J. W. (1996) Biotechniques 20: 266-276.
Steullet, V., Edwards-Bennett, S. & Dixon, D. W. (1999) Bioorg. Med. Chem. 7: 2531-2540.
Stuart, G. R. & Chambers, R. W. (1987) Nucleic Acids Res. 15: 7451-7462.
Wada, Y., Mitsumori, K, Terachi, T. & Ogawa, O. (1999) J. Mass Spectrom. 34: 885-888.
White, B. A. (ed.) (1993) PCR protocols. Humana Press, Totowa, N.J.
Zirvi, M., Bergstrom, D. E., Saurage, A. S., Hammer, R. P. & Barany, F. (1999a) Nucleic Acids Res. 27: e40.
Zirvi, M., Nakayama, T., Newman, G., McCaffrey, T., Paty, P. & Barany, F. (1999b) Nucleic Acids Res. 27: e41.
U.S. Pat. No. 6,197,498 March, 2001 Koster
U.S. Pat. No. 6,054,564 April, 2000 Barany, et al.
U.S. Pat. No. 5,869,242 February, 1999 Kamb
U.S. Pat. No. 4,683,195 July, 1987 Mullis et al.
WO 98/03673
EP 956359

1. A method for genotyping different alleles of a microsatellite DNA locus by using enzymatic and/or chemical agents that produce short, single-stranded DNA fragments of a size suitable for mass spectrometry analysis, said method comprising:
(a) providing a genomic DNA sample containing the microsatellite DNA;
(b) performing PCR amplification of a microsatellite DNA marker locus, using:
(i) an appropriate combinations of oligonucleotides
(ii) a dNTP mix in which the 2′-thymidine 5′-triphosphate is replaced by 2′-uridine 5′-triphosphate
(iii) a thermostable DNA polymerase that is capable of incorporating uridine nucleotides at positions where thymidine nucleotides are usually incorporated
(iv) an appropriate buffer
(c) treating the PCR fragment with Uracyl-DNA-Glycosylase
(d) treating further the UDG treated DNA with an enzymatic or chemical agent that cleaves DNA at abasic sites to yield single-stranded DNA products.

2. The method according to claim 1, wherein the genomic DNA sample is a mixture of different genomic DNAs.

3. The method according to claim 1, wherein the microsatellite DNA comprises mono-, di-, tri-, tetra-, penta-, hexa-, hepta-, octa- or nona-nucleotide repeated alleles.

4. The method according to claim 1, wherein the thermostable DNA polymerase is the Taq DNA polymerase.

5. The method according to claim 1, wherein the enzymatic agent that cleaves DNA at abasic sites is an AP-endonuclease.

6. The method according to claim 1, wherein the chemical agent that cleaves DNA at abasic sites is piperidine.

7. The method according to claim 1, wherein the chemical agent that cleaves DNA at abasic sites is a strong base.

8. The method according to claim 1, wherein [α-³²P]dNTP is added to the PCR reaction mix.

9. The method according to claim 1, further including a detecting step comprising separating said single-stranded DNA products according to their size by gel electrophoresis.

10. The method according to claim 1, further including a detecting step comprising separating the single-stranded DNA fragments according to their size by mass spectrometry.

11. A method for genotyping different alleles of a microsatellite DNA locus by using enzymatic and/or chemical agents that produce short, single-stranded DNA fragments of a size suitable for mass spectrometry analysis, said method comprising:
(a) providing a genomic DNA sample containing the microsatellite DNA;
(b) performing the PCR amplification of a microsatellite DNA marker locus, using:
(i) an appropriate combination of oligonucleotides
(ii) a conventional dNTP mix
(iii) a thermostable DNA polymerase that is capable of incorporating nucleotides at complementary positions on amplified DNA fragments; and,
(iv) an appropriate buffer;
(c) treating the PCR fragment with an agent selected from the group consisting of dimethyl sulfate, hydrazine in the presence of salts and hydrazine in the absence of salts
(d) treating further the treated DNA with an enzymatic or chemical agent that cleaves DNA at abasic sites to yield single stranded DNA products.

12. The method according to claim 11, wherein the treatment at step (c) comprises treatment with dimethyl sulfate (DMS).

13. The method according to claim 11, wherein the treatment at step (c) comprises treatment with hydrazine in the presence or absence of salts.

14. The method according to claim 11, wherein the chemical agent that cleaves DNA at abasic sites is piperidine.

15. The method according to claim 11, wherein the chemical agent that cleaves DNA at abasic sites is a strong base.

16. The method according to claim 11, wherein [α-³²P]dNTP is added to the PCR reaction mix.

17. The method according to claim ii, further including a detecting step comprising separating said single-stranded DNA products according to their size by gel electrophoresis.

18. The method according to claim 11, further including a detecting step comprising separating the single-stranded DNA fragments according to their size by mass spectrometry.

19. A kit for use in the detection of the presence of different microsatellite alleles at a locus on a DNA sample comprising:
(a) a container;
(b) combinations of appropriate oligonucleotides in said container which hybridize with target nucleotide sequences associated with said locus;
(c) a modified dNTP mix characterized in that all of one conventional base pair member in said dNTP mix are replaced with a substitute base pair member;
(d) DNA polymerase that is capable of incorporating nucleotides containing said substitute base pair member at positions where nucleotides containing said conventional base pair member would conventionally be incorporated;
(e) an agent that is capable of removing said substitute base pair member from DNA containing said base pair member; and
(f) an agent capable of cleaving DNA at abasic sites.

20. The kit of claim 19 further comprising [α-³²P]dNTP

21. A kit for use in the detection of the presence of different microsatellite alleles at a locus on a DNA sample comprising:
(a) a container;
(b) combinations of appropriate oligonucleotides in said container which hybridize with target nucleotide sequences associated with said locus
(c) a modified dNTP mix characterized in that 2′-thymidine 5′-triphosphate is replaced by 2′-uridine 5′-triphosphate
(d) a thermostable DNA polymerase that is capable of incorporating uridine nucleotides at positions where thymidine nucleotides are usually incorporated
(e) Uracyl-DNA-Glycosylase
(f) an agent that cleaves DNA at abasic sites.

22. The kit of claim 21 further comprising [α-³²P]dNTP

23. A kit for detection of the presence of different microsatellite alleles at a locus on a DNA sample comprising:
(a) a container;
(b) combinations of appropriate oligonucleotides in said container which hybridize with target nucleotide sequences associated with said locus;
(c) a conventional dNTP mix
(d) DNA polymerase
(e) an agent selected from the group consisting of dimethyl sulfate, hydrazine in the presence of salts, and hydrazine in the absence of salts; and,
(f) an agent capable of cleaving DNA at abasic sites.

24. A method for producing at least one single stranded DNA fragment containing a target microsatellite DNA locus, said fragment of a suitable size for mass spectrometry analysis, said method comprising:
(a) providing DNA material from a genome containing said microsatellite DNA locus;
(b) performing PCR amplification of said microsatellite DNA locus wherein said PCR amplification includes contacting said DNA material with
i) a modified dNTP mix characterized in that all of one conventional base pair member in said dNTP mix are replaced with a substitute base pair member; and,
ii) DNA polymerase that is capable of incorporating nucleotides containing said substitute base pair member at positions where nucleotides containing said conventional base pair member would conventionally be incorporated to obtain at least one PCR fragment containing said genetic marker locus;
(c) contacting said at least one PCR fragment from step (b) with an agent that removes said substitute base pair member from said PCR fragment to form at least one abasic site on said PCR fragment; and,
(d) contacting said PCR fragment from step (c) with an agent that cleaves DNA at abasic sites.

25. A method for producing at least one single stranded DNA fragment containing a target microsatellite DNA locus, said fragment of a suitable size for mass spectrometry analysis, said method comprising:
(1) Generating abasic sites in place of selected nucleotides in a PCR fragment containing a target microsatellite DNA locus wherein said target microsatellite DNA locus does not contain said selected nucleotides on one of its complementary DNA strands; and,
(2) contacting the PCR fragment containing said generated abasic sites with an agent that cleaves DNA at abasic sites.
